(19) United States
(12) Patent Application Publication
(10) Pub. No.: US 2010/0120926 A1
(43) Pub. Date: May 13, 2010

(54) Processes for Gasification of a Carbonaceous Feedstock

(75) Inventors: Earl T. Robinson, Lakeland, FL (US); Avinash Sirdeshpande, Chicago, IL (US); Eli Gal, Cupertino, CA (US); Vincent G. Reiling, Wheaton, IL (US); Nicholas Charles Nahas, Chatham, NJ (US)

Correspondence Address: MCDONNELL BOEHNEN HULBERT & BERGHOFF LLP, 300 S. WACKER DRIVE, SUITE 3100, CHICAGO, IL, 60606, US

(73) Assignee: GREATPOINT ENERGY, INC., Chicago, IL (US)

(21) Appl. No.: 12/562,921

(22) Filed: Sep. 18, 2009

Related U.S. Application Data
(60) Provisional application No. 61/098,472, filed on Sep. 19, 2008.

(51) Int. Cl. C07C 1/04

(52) U.S. Cl. 518/703

(57) ABSTRACT

The present invention relates to processes for preparing gaseous products, and in particular, methane via the catalytic gasification of carbonaceous feedstocks in the presence of steam. The processes comprise using at least one methanation step to convert carbon monoxide and hydrogen in the gaseous products to methane and do not recycle carbon monoxide or hydrogen to the catalytic gasifier.

### CROSS REFERENCE TO RELATED APPLICATION

This application claims priority under 35 U.S.C. §119 from U.S. Provisional Application Ser. No. 61/098,472 (filed Sep. 19, 2008), the disclosure of which is incorporated by reference herein for all purposes as if fully set forth.

This application is related to commonly owned and concurrently filed U.S. patent application Ser. No. ______, attorney docket no. FN-0039 US NP1, entitled CHAR METHANATION CATALYST AND ITS USE IN GASIFICATION PROCESSES.

### FIELD OF THE INVENTION

The present invention relates to processes for preparing gaseous products, and in particular, methane via the catalytic gasification of carbonaceous feedstocks in the presence of steam.

### BACKGROUND OF THE INVENTION

In view of numerous factors such as higher energy prices and environmental concerns, the production of value-added gaseous products from lower-fuel-value carbonaceous feedstocks, such as petroleum coke and coal, is receiving renewed attention. The catalytic gasification of such materials to produce methane and other value-added gases is disclosed, for example, in U.S. Pat. No. 3,828,474, U.S. Pat. No. 3,998,607, U.S. Pat. No. 4,057,512, U.S. Pat. No. 4,092,125, U.S. Pat. No. 4,094,650, U.S. Pat. No. 4,204,843, U.S. Pat. No. 4,468,231, U.S. Pat. No. 4,500,323, U.S. Pat. No. 4,541,841, U.S. Pat. No. 4,551,155, U.S. Pat. No. 4,558,027, U.S. Pat. No. 4,606,105, U.S. Pat. No. 4,617,027, U.S. Pat. No. 4,609,456, U.S. Pat. No. 5,017,282, U.S. Pat. No. 5,055,181, U.S. Pat. No. 6,187,465, U.S. Pat. No. 6,790,430, U.S. Pat. No. 6,894,183, U.S. Pat. No. 6,955,695, US2003/0167961A1, US2006/0265953A1, US2007/000177A1, US2007/083072A1, US2007/0277437A1, US2009/0048476A1, US2009/0090056A1, US2009/0090055A1, US2009/0165383A1, US2009/0166588A1, US2009/0165379A1, US2009/0170968A1, US2009/0165380A1, US2009/0165381A1, US2009/0165361A1, US2009/0165382A1, US2009/0169449A1, US2009/0169448A1, US2009/0165376A1, US2009/0165384A1, US2009/0217584A1, US2009/0217585A1, US2009/0217590A1, US2009/0217586A1, US2009/0217588A1, US2009/0217589A1, US2009/0217575A1, US2009/0217587A1 and GB1599932.

In general, carbonaceous materials, such as coal or petroleum coke, can be converted to a plurality of gases, including value-added gases such as methane, by the gasification of the material in the presence of an alkali metal catalyst source and steam at elevated temperatures and pressures. Fine unreacted carbonaceous materials are removed from the raw gases produced by the gasifier, the gases are cooled and scrubbed in multiple processes to remove undesirable contaminants and other side-products including carbon monoxide, hydrogen, carbon dioxide, and hydrogen sulfide.

In order to maintain the net heat of reaction as close to neutral as possible (only slightly exothermic or endothermic; i.e., that the reaction is run under thermally neutral conditions) a recycle carbon monoxide and hydrogen gas stream is often fed to the catalytic gasifiers. See, for example, U.S. Pat. No. 4,094,650, U.S. Pat. No. 6,955,595 and US2007/083072A1. Such gas recycle loops generally require at least additional heating elements and pressurization elements to bring the recycle gas stream to a temperature and pressure suitable for introduction into the catalytic gasifier. Further, such processes for generating methane can require separation of methane from the recycle gases, for example, by cryogenic distillation. In doing so, the engineering complexity and overall cost of producing methane is greatly increased.

Therefore, a need remains for improved gasification processes where gas recycle loops are minimized and/or eliminated to decrease the complexity and cost of producing methane.

### SUMMARY OF THE INVENTION

In one aspect, the invention provides a process for generating a plurality of gaseous products from a carbonaceous feedstock, and recovering a methane product stream, the process comprising the steps of:

(a) supplying methane, an oxygen-rich gas and steam to a thermal reformer, the reformer in communication with a gasifier;

(b) reforming a substantial portion of the methane supplied to the thermal reformer, in the presence of the oxygen-rich gas and under suitable temperature and pressure, to generate a first gas stream comprising hydrogen, carbon monoxide and superheated steam;

(c) introducing a carbonaceous feedstock, a gasification catalyst and the first gas stream to a gasifier;

(d) reacting the carbonaceous feedstock and the first gas stream in the gasifier in the presence of the gasification catalyst under suitable temperature and pressure to form a second gas stream comprising a plurality of gaseous products comprising methane, carbon dioxide, hydrogen, carbon monoxide and hydrogen sulfide;

(e) optionally reacting at least a portion of the carbon monoxide and at least a portion of the hydrogen in the second gas stream in a catalytic methanator in the presence of a sulfur-tolerant methanation catalyst to produce a methane-enriched second gas stream;

(f) removing a substantial portion of the carbon dioxide and a substantial portion of the hydrogen sulfide from the second gas stream (or the methane-enriched second gas stream if present) to produce a third gas stream comprising a substantial portion of the methane from the second gas stream (or the methane-enriched second gas stream if present);

(g) optionally, if the third gas stream comprises hydrogen and greater than about 100 ppm carbon monoxide, reacting the carbon monoxide and hydrogen present in the third gas stream in a catalytic methanator in the presence of a methanation catalyst to produce a methane-enriched third gas stream; and

(h) recovering the third gas stream (or the methane-enriched third gas stream if present),

wherein (i) at least one of step (e) and step (g) is present, and (ii) the third gas stream (or the methane-enriched third gas stream if present) is the methane product stream, or the third gas stream (or the methane-enriched third gas stream if present) is purified to generate the methane product stream.

In a second aspect, the invention provides a continuous process for generating a plurality of gaseous products from a carbonaceous feedstock, and recovering a methane product stream, the process comprising the steps of:

(a) continuously supplying methane, an oxygen-rich gas stream and steam to a thermal reformer, the reformer in communication with a catalytic gasifier;

(b) continuously reforming a substantial portion of the methane supplied to the thermal reformer, in the presence of the oxygen-rich gas stream and under suitable temperature and pressure, to generate a first gas stream comprising hydrogen, carbon monoxide and superheated steam;

(c) continuously introducing a carbonaceous feedstock, a gasification catalyst and the first gas stream to a catalytic gasifier;

(d) continuously reacting the carbonaceous feedstock and the first gas stream in the catalytic gasifier in the presence of the gasification catalyst under suitable temperature and pressure to form a second gas stream comprising a plurality of gaseous products comprising methane, carbon dioxide, hydrogen, carbon monoxide and hydrogen sulfide;

(e) optionally reacting at least a portion of the carbon monoxide and at least a portion of the hydrogen present in the second gas stream in a catalytic methanator in the presence of a sulfur-tolerant methanation catalyst to produce a methane-enriched second gas stream;

(f) continuously removing a substantial portion of the carbon dioxide and a substantial portion of the hydrogen sulfide from the second gas stream (or the methane-enriched second gas stream if present) to produce a third gas stream comprising a substantial portion of the methane from the second gas stream (or the methane-enriched second gas stream if present);

(g) optionally, if the third gas stream comprises hydrogen and greater than about 100 ppm carbon monoxide, reacting the carbon monoxide and hydrogen present in the third gas stream in a catalytic methanator in the presence of a methanation catalyst to produce a methane-enriched third gas stream; and

(h) continuously recovering the third gas stream (or the methane-enriched third gas stream if present),

wherein (i) at least one of step (e) and step (g) is present, and (ii) the third gas stream (or the methane-enriched third gas stream if present) is the methane product stream, or the third gas stream (or the methane-enriched third gas stream if present) is purified to generate the methane product stream.

The processes in accordance with the present invention can be useful, for example, for producing methane from various carbonaceous feedstocks. A preferred process is one which produces a product stream of “pipeline-quality natural gas” as described in further detail below.

### BRIEF DESCRIPTION OF THE DRAWINGS

FIG. 1 is a diagram of an embodiment of a gasification process comprising a thermal reformer and steam source to supply superheated steam and syngas to a catalytic gasifier and a methanator downstream of acid gas removal processes.

FIG. 2 is a diagram of an embodiment of a gasification process comprising a thermal reformer and steam source to supply superheated steam and syngas to a catalytic gasifier and a sulfur-tolerant methanator upstream of acid gas removal operations and an optional trim methanator downstream of the acid gas removal processes.

FIG. 3 is a diagram of another embodiment of a gasification process where the methane provided to the thermal reformer in the embodiment of FIG. 1 is optionally a portion of the methane product stream or second gas stream from the acid gas removal processes.

FIG. 4 is a diagram of another embodiment of a gasification process where the methane provided to the thermal reformer in the embodiment of FIG. 1 is a portion of the methane product stream, the third gas stream or both from the acid gas removal processes. At least a portion of the char can be optionally recycled as a sulfur tolerant methanation catalyst. An optional trim methanator downstream of the acid gas removal processes can be used.

FIG. 5 is a diagram of another embodiment of a gasification process comprising the processes of FIG. 3 in combination with processes for preparing the catalyzed feedstock and recovering and recycling catalyst from the char produced by the catalytic gasifier. At least a portion of the gas stream downstream from the methanation step can recycled into the thermal reformer.

FIG. 6 is a diagram of another embodiment of a gasification process comprising the processes of FIG. 4 in combination with processes for preparing the catalyzed feedstock, recovering and recycling catalyst from the char produced by the catalytic gasifier, and optionally utilizing a portion of the char from the catalytic gasifier as a sulfur-tolerant catalyst in the sulfur-tolerant methanator. An optional trim methanation step can be included downstream of the acid gas removal step.

### DETAILED DESCRIPTION

The present disclosure relates to processes to convert a carbonaceous feedstock into a plurality of gaseous products including at least methane, the processes comprising, among other steps, providing methane and steam to a thermal reformer (e.g., an autothermal reformer or a partial oxidation reactor) to generate carbon monoxide, hydrogen and superheated steam for introduction to a gasifier to convert the carbonaceous feedstock in the presence of an alkali metal catalyst into the plurality of gaseous products. In particular, the present invention provides improved gasification processes where there advantageously can be no recycle of carbon monoxide or hydrogen to the gasifier. The carbon monoxide and hydrogen input desirable for near-equilibrium operation of the catalytic gasification can be supplied instead by the thermal reformer. The superheated steam used in the catalytic gasification can also be provided by the thermal reformer.

A “methane-containing gas stream” as used herein refers to a gas stream containing at least about 50 mol % methane. In some cases, the methane-containing gas stream will contain at least about 66 mol % methane, or at least about 75 mol % methane. In some cases, the methane-containing gas stream will contain at least about 90 mol %, or at least about 95 mol %, combined of methane, hydrogen and carbon monoxide. Such methane-containing gas streams are provided to a thermal reformer as discussed below.

The present invention can be practiced in conjunction with the subject matter disclosed in commonly-owned US2007/0000177A1, US2007/0083072A1, US2007/0277437A1, US2009/0048476A1, US2009/0090056A1, US2009/0090055A1, US2009/0165383A1, US2009/0166588A1, US2009/0165379A1, US2009/0170968A1, US2009/0165380A1, US2009/0165381A1, US2009/0165361A1, US2009/0165382A1, US2009/0169449A1, US2009/0169448A1, US2009/0165376A1, US2009/0165384A1, US2009/0217582A1, US2009/0220406A1, US2009/0217590A1, US2009/0217586A1, US2009/0217588A1, US2009/0218424A1, US2009/0217589A1, US2009/0217575A1 and US2009/0217587A1.

Moreover, the present invention can be practiced in conjunction with the subject matter disclosed in commonly-owned U.S. patent application Ser. Nos. 12/395,330 and 12/395,433, each of which was filed 27 Feb. 2009; 12/415,042 and 12/415,050, each of which was filed 31 Mar. 2009; and 12/492,467, 12/492,477, 12/492,484, 12/492,489 and 12/492,497, each of which was filed 26 Jun. 2009.

Further, the present invention can be practiced using developments described in previously incorporated U.S. patent application Ser. No. ______, attorney docket no. FN-0039 US NP1, entitled CHAR METHANATION CATALYST AND ITS USE IN GASIFICATION PROCESSES.

All publications, patent applications, patents and other references mentioned herein, if not otherwise indicated, are explicitly incorporated by reference herein in their entirety for all purposes as if fully set forth.

Unless otherwise defined, all technical and scientific terms used herein have the same meaning as commonly understood by one of ordinary skill in the art to which this disclosure belongs. In case of conflict, the present specification, including definitions, will control.

Except where expressly noted, trademarks are shown in upper case.

Although processes and materials similar or equivalent to those described herein can be used in the practice or testing of the present disclosure, suitable processes and materials are described herein.

Unless stated otherwise, all percentages, parts, ratios, etc., are by weight.

When an amount, concentration, or other value or parameter is given as a range, or a list of upper and lower values, this is to be understood as specifically disclosing all ranges formed from any pair of any upper and lower range limits, regardless of whether ranges are separately disclosed. Where a range of numerical values is recited herein, unless otherwise stated, the range is intended to include the endpoints thereof, and all integers and fractions within the range. It is not intended that the scope of the present disclosure be limited to the specific values recited when defining a range.

When the term “about” is used in describing a value or an end-point of a range, the disclosure should be understood to include the specific value or end-point referred to.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but can include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The use of “a” or “an” to describe the various elements and components herein is merely for convenience and to give a general sense of the disclosure. This description should be read to include one or at least one and the singular also includes the plural unless it is obvious that it is meant otherwise.

The term “substantial portion”, as used herein, unless otherwise defined herein, means that greater than about 90% of the referenced material, preferably greater than 95% of the referenced material, and more preferably greater than 97% of the referenced material. The percent is on a molar basis when reference is made to a molecule (such as methane, carbon dioxide, carbon monoxide and hydrogen sulfide), and otherwise is on a weight basis (such as for entrained carbonaceous fines).

The term “carbonaceous material” as used herein can be, for example, biomass and non-biomass materials as defined herein.

The term “biomass” as used herein refers to carbonaceous materials derived from recently (for example, within the past 100 years) living organisms, including plant-based biomass and animal-based biomass. For clarification, biomass does not include fossil-based carbonaceous materials, such as coal. For example, see previously incorporated US2009/0217575A1 and US2009/0217587A1.

The term “plant-based biomass” as used herein means materials derived from green plants, crops, algae, and trees, such as, but not limited to, sweet sorghum, bagasse, sugarcane, bamboo, hybrid poplar, hybrid willow, albizia trees, eucalyptus, alfalfa, clover, oil palm, switchgrass, sudangrass, millet, jatropha, and miscanthus (e.g., Miscanthus×giganteus). Biomass further include wastes from agricultural cultivation, processing, and/or degradation such as corn cobs and husks, corn stover, straw, nut shells, vegetable oils, canola oil, rapeseed oil, biodiesels, tree bark, wood chips, sawdust, and yard wastes.

The term “animal-based biomass” as used herein means wastes generated from animal cultivation and/or utilization. For example, biomass includes, but is not limited to, wastes from livestock cultivation and processing such as animal manure, guano, poultry litter, animal fats, and municipal solid wastes (e.g., sewage).

The term “non-biomass”, as used herein, means those carbonaceous materials which are not encompassed by the term “biomass” as defined herein. For example, non-biomass include, but is not limited to, anthracite, bituminous coal, sub-bituminous coal, lignite, petroleum coke, asphaltenes, liquid petroleum residues or mixtures thereof. For example, see previously incorporated US2009/0166588A1, US2009/0165379A1, US2009/0165380A1, US2009/0165361A1, US2009/0217590A1 and US2009/0217586A1.

The terms “petroleum coke” and “petcoke” as used here includes both (i) the solid thermal decomposition product of high-boiling hydrocarbon fractions obtained in petroleum processing (heavy residues—“resid petcoke”); and (ii) the solid thermal decomposition product of processing tar sands (bituminous sands or oil sands—“tar sands petcoke”). Such carbonization products include, for example, green, calcined, needle and fluidized bed petcoke.

Resid petcoke can also be derived from a crude oil, for example, by coking processes used for upgrading heavy-gravity residual crude oil, which petcoke contains ash as a minor component, typically about 1.0 wt % or less, and more typically about 0.5 wt % of less, based on the weight of the coke. Typically, the ash in such lower-ash cokes comprises metals such as nickel and vanadium.

Tar sands petcoke can be derived from an oil sand, for example, by coking processes used for upgrading oil sand. Tar sands petcoke contains ash as a minor component, typically in the range of about 2 wt % to about 12 wt %, and more typically in the range of about 4 wt % to about 12 wt %, based on the overall weight of the tar sands petcoke. Typically, the ash in such higher-ash cokes comprises materials such as silica and/or alumina.

Petroleum coke has an inherently low moisture content, typically, in the range of from about 0.2 to about 2 wt % (based on total petroleum coke weight); it also typically has a very low water soaking capacity to allow for conventional catalyst impregnation methods. The resulting particulate compositions contain, for example, a lower average moisture content which increases the efficiency of downstream drying operation versus conventional drying operations.

The petroleum coke can comprise at least about 70 wt % carbon, at least about 80 wt % carbon, or at least about 90 wt % carbon, based on the total weight of the petroleum coke. Typically, the petroleum coke comprises less than about 20 wt % inorganic compounds, based on the weight of the petroleum coke.

The term “asphaltene” as used herein is an aromatic carbonaceous solid at room temperature, and can be derived, from example, from the processing of crude oil and crude oil tar sands.

The term “coal” as used herein means peat, lignite, sub-bituminous coal, bituminous coal, anthracite, or mixtures thereof. In certain embodiments, the coal has a carbon content of less than about 85%, or less than about 80%, or less than about 75%, or less than about 70%, or less than about 65%, or less than about 60%, or less than about 55%, or less than about 50% by weight, based on the total coal weight. In other embodiments, the coal has a carbon content ranging up to about 85%, or up to about 80%, or up to about 75% by weight, based on the total coal weight. Examples of useful coal include, but are not limited to, Illinois #6, Pittsburgh #8, Beulah (ND), Utah Blind Canyon, and Powder River Basin (PRB) coals. Anthracite, bituminous coal, sub-bituminous coal, and lignite coal may contain about 10 wt %, from about 5 to about 7 wt %, from about 4 to about 8 wt %, and from about 9 to about 11 wt %, ash by total weight of the coal on a dry basis, respectively. However, the ash content of any particular coal source will depend on the rank and source of the coal, as is familiar to those skilled in the art. See, for example, “Coal Data: A Reference”, Energy Information Administration, Office of Coal, Nuclear, Electric and Alternate Fuels, U.S. Department of Energy, DOE/EIA-0064(93), February 1995.

The ash produced from a coal typically comprises both a fly ash and a bottom ash, as are familiar to those skilled in the art. The fly ash from a bituminous coal can comprise from about 20 to about 60 wt % silica and from about 5 to about 35 wt % alumina, based on the total weight of the fly ash. The fly ash from a sub-bituminous coal can comprise from about 40 to about 60 wt % silica and from about 20 to about 30 wt % alumina, based on the total weight of the fly ash. The fly ash from a lignite coal can comprise from about 15 to about 45 wt % silica and from about 20 to about 25 wt % alumina, based on the total weight of the fly ash. See, for example, Meyers, et al. “Fly Ash. A Highway Construction Material.” Federal Highway Administration, Report No. FHWA-IP-76-16, Washington, D.C., 1976.

The bottom ash from a bituminous coal can comprise from about 40 to about 60 wt % silica and from about 20 to about 30 wt % alumina, based on the total weight of the bottom ash. The bottom ash from a sub-bituminous coal can comprise from about 40 to about 50 wt % silica and from about 15 to about 25 wt % alumina, based on the total weight of the bottom ash. The bottom ash from a lignite coal can comprise from about 30 to about 80 wt % silica and from about 10 to about 20 wt % alumina, based on the total weight of the bottom ash. See, for example, Moulton, Lyle K. “Bottom Ash and Boiler Slag,” Proceedings of the Third International Ash Utilization Symposium. U.S. Bureau of Mines, Information Circular No. 8640, Washington, D.C., 1973.

The term “unit” refers to a unit operation. When more than one “unit” is described as being present, those units are operated in a parallel fashion. A single “unit”, however, may comprise more than one of the units in series. For example, an acid gas removal unit may comprise a hydrogen sulfide removal unit followed in series by a carbon dioxide removal unit. As another example, a trace contaminant removal unit may comprise a first removal unit for a first trace contaminant followed in series by a second removal unit for a second trace contaminant. As yet another example, a methane compressor unit may comprise a first methane compressor to compress the methane product stream to a first pressure, followed in series by a second methane compressor to further compress the methane product stream to a second (higher) pressure.

The materials, processes, and examples herein are illustrative only and, except as specifically stated, are not intended to be limiting.

Gasification Processes

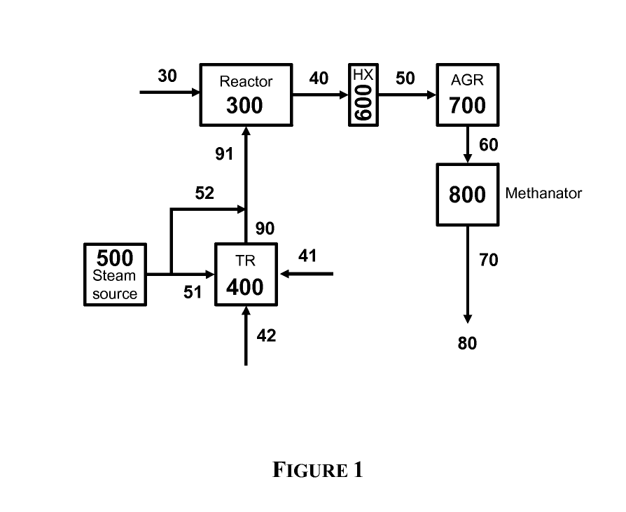

In one embodiment of the invention, a methane product stream (80) can be generated from a catalyzed carbonaceous feedstock (30) as illustrated in FIG. 1. A first portion of steam (51) from a steam source (500), an oxygen-rich gas (42) such as purified oxygen, and methane (41) can be provided to a thermal reformer (400) to generate a hot gas stream (90) comprising superheated steam, carbon monoxide and hydrogen at a temperature above the operating temperature of reactor (300) sufficient to maintain the thermal balance in reactor (300), as discussed in further detail below. The hot gas stream (90) can be combined with a second portion of steam (52) from the steam source to generate a first gas stream (91) comprising carbon monoxide, hydrogen, and superheated steam. By utilizing such a process, the use of a superheater to generate the superheated steam for providing the catalytic gasifier, as disclosed in many of the previously incorporated references, can be eliminated.

The thermal reformer generates carbon monoxide and hydrogen from methane in the presence of an oxidizing gas. Examples of thermal reformers include, but are not limited to autothermal reformers (ATRs), steam methane reformers (SMRs), and partial oxidation reactors (POx). Steam methane reformers react steam and methane at high temperatures and moderate pressures over a reduced nickel-containing catalyst to produce synthesis gas where the reaction heat is applied externally to the process. Partial oxidation reactors (POx) utilize oxygen to generate hydrogen, carbon monoxide, and carbon dioxide from partial combustion of a hydrocarbon containing feed source, such as methane.

Autothermal reformers combine catalytic partial oxidation and steam reforming. Partial oxidation employs substoichiometric combustion of a hydrocarbon fuel (e.g., methane) to achieve the temperatures to reform the fuel. In the overall process, fuel, oxidant (oxygen or air, for example), and steam are reacted to form primarily hydrogen, CO₂ and CO. The exothermic combustion reactions drive the endothermic reforming reaction. Steam and/or oxygen addition can be staged to provide control of the carbon monoxide:hydrogen ratio of the hot gas stream (90) and therefore the first gas stream (91). In certain embodiments, the hydrogen and carbon monoxide in the first gas stream are present in a molar ratio of about 3:1. Autothermal reformers typically employ nickel- or noble metal-based catalyst beds, as are familiar to those skilled in the art, and operate at temperatures up to about 2300° F. (e.g., 1600-2300° F.). ATRs are commercially available from companies such as Haldor Topsoe A/S (Lyngby, Denmark) and HyRadix (Des Plaines, Ill.).

Any of the steam boilers known to those skilled in the art can supply steam for the thermal reformer (400) and/or for mixing with the hot gas stream (90) generated by the thermal reformer. Such boilers can be powered, for example, through the use of any carbonaceous material such as powdered coal, biomass etc., and including but not limited to rejected carbonaceous materials from the feedstock preparation operations (e.g., fines, supra). Steam can also be supplied from an additional catalytic gasifier coupled to a combustion turbine where the exhaust from the reactor is thermally exchanged to a water source and produce steam. Alternatively, the steam may be generated for the catalytic gasifiers as described in previously incorporated US2009/0165376A1, US2009/0217584A1 and US2009/0217585A1.

Steam recycled or generated from other process operations can also be used as a sole steam source, or in combination with the steam from a steam generator to supply steam to the thermal reformer (400) or for mixing with the hot gas stream (90) or provided directly to the catalytic gasification process. For example, when the slurried carbonaceous materials are dried with a fluid bed slurry drier, as discussed below for the preparation of the catalyzed carbonaceous feedstock (30), the steam generated through vaporization can be fed to the thermal reformer (400) or mixed with the hot gas stream (90) or provided directly to the catalytic gasification process. Further, steam generated by a heat exchanger unit (such as 600) can be fed to the thermal reformer (400) or used for mixing with the hot gas stream (90) or provided directly to the catalytic gasification process.

The catalyzed carbonaceous feedstock (30) can be provided to a catalytic gasifier (300) in the presence of the first gas stream (91) and under suitable pressure and temperature conditions to generate a second gas stream (40) comprising a plurality of gaseous products comprising methane, carbon dioxide, hydrogen, carbon monoxide, and hydrogen sulfide. The catalyzed carbonaceous feedstock (30) typically comprises one or more carbonaceous materials and one or more gasification catalysts, as discussed below.

The catalytic gasifiers for such processes are typically operated at moderately high pressures and temperature, requiring introduction of the catalyzed carbonaceous feedstock (30) to a reaction chamber of the catalytic gasifier while maintaining the required temperature, pressure, and flow rate of the feedstock. Those skilled in the art are familiar with feed inlets to supply the catalyzed carbonaceous feedstock into the reaction chambers having high pressure and/or temperature environments, including, star feeders, screw feeders, rotary pistons, and lock-hoppers. It should be understood that the feed inlets can include two or more pressure-balanced elements, such as lock hoppers, which would be used alternately. In some instances, the catalyzed carbonaceous feedstock can be prepared at pressures conditions above the operating pressure of catalytic gasifier. Hence, the particulate composition can be directly passed into the catalytic gasifier without further pressurization.

Any of several types of catalytic gasifiers can be utilized. Suitable catalytic gasifiers include those having a reaction chamber which is a counter-current fixed bed, a co-current fixed bed, a fluidized bed, or an entrained flow or moving bed reaction chamber.

Gasification in the catalytic gasifier is typically affected at moderate temperatures of at least about 450° C., or of at least about 600° C., or of at least about 650° C., to about 900° C., or to about 800° C., or to about 750° C.; and at pressures of at least about 50 psig, or at least about 200 psig, or at least about 400 psig, to about 1000 psig, or to about 700 psig, or to about 600 psig.

The gas utilized in the catalytic gasifier for pressurization and reactions of the particulate composition can comprise, for example, the first gas stream, and/or optionally, additional steam, oxygen, nitrogen, air, or inert gases such as argon which can be supplied to the catalytic gasifier according to methods known to those skilled in the art. As a consequence, the first gas stream must be provided at a higher pressure which allows it to enter the catalytic gasifier.

The catalytic conversion of a carbon source to methane that occurs in the catalytic gasifier typically involves three separate reactions:

Steam carbon: C+H₂O→CO+H₂ (I)

Water-gas shift: CO+H₂O→H₂+CO₂ (II)

CO Methanation: CO+3H₂→CH₄+H₂O (III)

These three reactions are together essentially thermally balanced; however, due to process heat losses and other energy requirements (such as required for evaporation of moisture entering the gasifier with the feedstock), some heat must be added to the catalytic gasifier to maintain the thermal balance. The superheating of the first gas stream to a temperature above the operating temperature of the catalytic gasifier, via the thermal reformer, can be the primary mechanism for supplying this extra heat. As mentioned previously, this allows the process to be configured without a separate superheater.

A person of ordinary skill in the art can determined the amount of heat required to be added to the catalytic gasifier to substantially maintain thermal balance. When considered in conjunction with flow rate and composition of the first gas stream (and other factors recognizable to those of ordinary skill in the relevant art), this will in turn dictate the temperature and pressure of the first gas stream as it enters the catalytic gasifier (and in turn the operating temperature and pressure of the autothermal reactor).

The hot gas effluent leaving the reaction chamber of the catalytic gasifier can pass through a fines remover unit portion of the catalytic gasifier which serves as a disengagement zone where particles too heavy to be entrained by the gas leaving the catalytic gasifier (i.e., fines) are returned to the reaction chamber (e.g., fluidized bed). The fines remover unit can include one or more internal and/or external cyclone separators or similar devices to remove fines and particulates from the hot gas effluent. The resulting second gas stream (40) leaving the catalytic gasifier generally comprises CH₄, CO₂, H₂, CO, H₂S, unreacted steam, entrained fines, and optionally, other contaminants such as NH₃, COS, HCN and/or elemental mercury vapor, depending on the nature of the carbonaceous material utilized for gasification.

Residual entrained fines may be substantially removed, when necessary, by any suitable device such as external cyclone separators optionally followed by Venturi scrubbers. The recovered fines can be processed to recover alkali metal catalyst, or directly recycled back to feedstock preparation as described in previously incorporated US2009/0217589A1.

Removal of a “substantial portion” of fines means that an amount of fines is removed from the hot first gas stream such that downstream processing is not adversely affected; thus, at least a substantial portion of fines should be removed. Some minor level of ultrafine material may remain in hot first gas stream to the extent that downstream processing is not significantly adversely affected. Typically, at least about 90 wt %, or at least about 95 wt %, or at least about 98 wt %, of the fines of a particle size greater than about 20 μm, or greater than about 10 μm, or greater than about 5 μm, are removed.

The second gas stream (40), upon exiting reactor (300), will typically comprise at least about 20 mol % methane based on the moles of methane, carbon dioxide, carbon monoxide and hydrogen in the second gas stream. In addition, the second gas stream will typically comprise at least about 50 mol % methane plus carbon dioxide, based on the moles of methane, carbon dioxide, carbon monoxide and hydrogen in the second gas stream.

The second gas stream (40) may be provided to a heat exchanger (600) to reduce the temperature of the second gas stream and generate a cooled second gas stream (50) having a temperature less than the second gas stream (40). The cooled second gas stream (50) can be provided to acid gas removal (AGR) processes (700) as described below.

Depending on gasification conditions, the second gas stream (40) can be generated having at a temperature ranging from about 450° C. to about 900° C. (more typically from about 650° C. to about 800° C.), a pressure of from about 50 psig to about 1000 psig (more typically from about 400 psig to about 600 psig), and a velocity of from about 0.5 ft/sec to about 2.0 ft/sec (more typically from about 1.0 ft/sec to about 1.5 ft/sec). The heat energy extracted by any one or more of the heat exchanger units (600), when present, can, for example, be used to generate steam, which can be utilized, for example, as a portion of the steam supplied to the thermal reformer (400) or for mixing with the hot gas stream (90), as discussed above. The resulting cooled second gas stream (50) will typically exit the heat exchanger (600) at a temperature ranging from about 250° C. to about 600° C. (more typically from about 300° C. to about 500° C.), a pressure of from about 50 psig to about 1000 psig (more typically from about 400 psig to about 600 psig), and a velocity of from about 0.5 ft/sec to about 2.5 ft/sec (more typically from about 1.0 ft/sec to about 1.5 ft/sec).

Subsequent acid gas removal processes (700) can be used to remove a substantial portion of H₂S and CO₂ from the cooled second gas stream (50) and generate a third gas stream (60). Acid gas removal processes typically involve contacting the cooled second gas stream (50) with a solvent such as monoethanolamine, diethanolamine, methyldiethanolamine, diisopropylamine, diglycolamine, a solution of sodium salts of amino acids, methanol, hot potassium carbonate or the like to generate CO₂ and/or H₂S laden absorbers. One method can involve the use of Selexol® (UOP LLC, Des Plaines, Ill. USA) or Rectisol® (Lurgi AG, Frankfurt am Main, Germany) solvent having two trains; each train consisting of an H₂S absorber and a CO₂ absorber.

The resulting third gas stream (60) can comprise CH₄, H₂, and, optionally, CO when the sour shift unit (infra) is not part of the process, and typically, small amounts of CO₂ and H₂O. One method for removing acid gases from the cooled second gas stream (50) is described in previously incorporated US2009/0220406A1.

At least a substantial portion (e.g., substantially all) of the CO₂ and/or H₂S (and other remaining trace contaminants) should be removed via the acid gas removal processes. “Substantial” removal in the context of acid gas removal means removal of a high enough percentage of the component such that a desired end product can be generated. The actual amounts of removal may thus vary from component to component. For “pipeline-quality natural gas”, only trace amounts (at most) of H₂S can be present, although higher amounts of CO₂ may be tolerable.

Typically, at least about 85%, or at least about 90%, or at least about 92%, of the CO₂, and at least about 95%, or at least about 98%, or at least about 99.5%, of the H₂S, should be removed from the cooled second gas stream (50).

Losses of desired product (methane) in the acid gas removal step should be minimized such that the third gas stream (60) comprises at least a substantial portion (and substantially all) of the methane from the cooled second gas stream (50). Typically, such losses should be about 2 mol % or less, or about 1.5 mol % or less, or about 1 mol % of less, of the methane from the cooled second gas stream (50).

The gasification processes described herein utilize at least one methanation step to generate methane from the carbon monoxide and hydrogen present in one or more of the second gas stream (e.g., hot second gas stream (40), and/or cooled second gas stream (50)), and third gas stream (60). For example, in one embodiment of the invention, at least a portion of the carbon monoxide and at least a portion of the hydrogen in the second gas stream is reacted in a catalytic methanator in the presence of a sulfur-tolerant methantion catalyst to produce a methane-enriched second gas stream, which can then be subjected to acid gas removal as described above (i.e., step (e) is performed). In other embodiments of the invention, if the third gas stream comprises hydrogen and greater than above 100 ppm carbon monoxide, carbon monoxide and hydrogen present in the third gas stream are reacted in a catalytic methanator in the presence of a methanation catalyst to produce a methane-enriched third gas stream (i.e., step (g) is performed). In certain embodiments of the invention, both of these methanation steps (i.e., steps (c) and (g) can be performed).

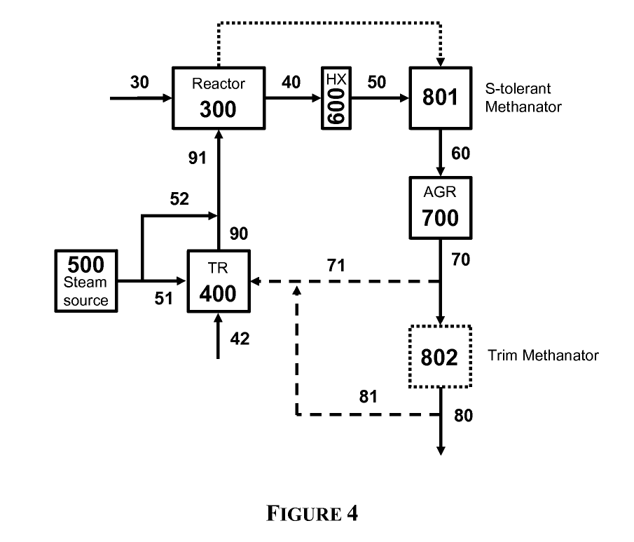

For example, in one embodiment, as shown in FIG. 1, the third gas stream (60) may be passed to a catalytic methanator (800) in which carbon monoxide and hydrogen present in the third gas stream (60) can be reacted to generate methane, thereby generating a methane-enriched third gas stream (70) (i.e., step (g) is present in the process). In various embodiments, the methane-enriched third gas stream (70) is the methane product stream (80). In various other embodiments, the methane-enriched third gas stream (70) can be further purified to generate the methane product stream (80). Further purifications processes include, but are not limited to, additional trim methanators (e.g., (802) in FIG. 4), cryogenic separators and membrane separators.

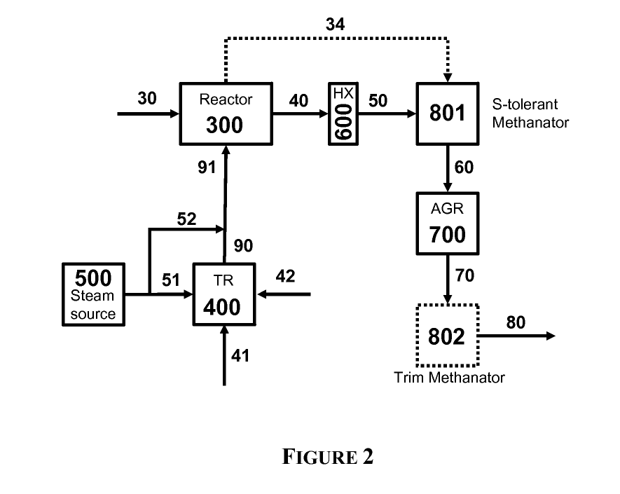

In another embodiment, as illustrated in FIG. 2, the second (40) or cooled second (50) gas stream can be passed to a sulfur-tolerant catalytic methanator (801) where carbon monoxide and hydrogen in the second (40) or cooled second (50) gas stream can be reacted to generate methane and thereby a methane-enriched second gas stream (60) (i.e., step (e) is present in the process). The second (40) or cooled second (50) gas streams typically contain significant quantities of hydrogen sulfide which can deactivate methanation catalysts as is familiar to those skilled in the art. Therefore, in such embodiments, the catalytic methanator (801) comprises a sulfur-tolerant methanation catalyst such as molybdenum and/or tungsten sulfides. Further examples of sulfur-tolerant methanation catalysts include, but are not limited to, catalysts disclosed in U.S. Pat. No. 4,243,554, U.S. Pat. No. 4,243,553, U.S. Pat. No. 4,006,177, U.S. Pat. No. 3,958,957, U.S. Pat. No. 3,928,000, US2490488; Mills and Steffgen, in Catalyst Rev. 8, 159 (1973), and Schultz et al, U.S. Bureau of Mines, Rep. Invest. No. 6974 (1967).

In one particular example, the sulfur-tolerant methanation catalyst is a portion of the char product (34) generated by the catalytic gasifier (300) which can be periodically removed from the catalytic gasifier (300) and transferred to the sulfur-tolerant catalytic methanator (801), as is described in previously incorporated U.S. patent application Ser. No. ______, attorney docket no. FN-0039 US NP1, entitled CHAR METHANATION CATALYST AND ITS USE IN GASIFICATION SYSTEMS. Operating conditions for a methanator utilizing the char can be similar to those set forth in previously incorporated U.S. Pat. No. 3,958,957. When one or more methanation steps are included in an integrated gasification process that employs at least a portion of the char product as the sulfer-tolerant methanation catalyst, e.g., such as the integrated gasification process shown in FIG. 4, the methanation temperatures generally range from about 450° C., or from about 475° C., or from about 500° C., to about 650° C., or to about 625° C., or to about 600° C. and at a pressure from about 400 to about 750 psig.

Any remaining portion of the char can be processed to recover and recycle entrained catalyst compounds, as discussed below.

Continuing with FIG. 2, the methane-enriched second gas stream (60) can be provided to a subsequent acid gas removal process (700), as described previously, to remove a substantial portion of H₂S and CO₂ from the methane-enriched second gas stream (60) and generate a third gas stream (70). In various embodiments, the third gas stream (70) can be the methane product stream (80).

In other embodiments, the third gas stream (70) can contain appreciable amounts of carbon monoxide and hydrogen. In such examples, the third gas stream (70) can be provided to a methanator (e.g., trim methanator (802)) in which carbon monoxide and hydrogen in the third gas stream (70) can be reacted, under suitable temperature and pressure conditions, to generate methane and thereby a methane-enriched third gas stream (80) (e.g., steps (e) and (g) as described above).

In a particular example, the third gas stream (70), when it contains appreciable amounts of CO (e.g., greater than about 100 ppm CO), can be further enriched in methane by performing trim methanation to reduce the CO content. One may carry out trim methanation using any suitable method and apparatus known to those of skill in the art, including, for example, the method and apparatus disclosed in U.S. Pat. No. 4,235,044, incorporated herein by reference.

### Examples of Specific Embodiments

As described in more detail below, in one embodiment of the invention, the gasification catalyst can comprise an alkali metal gasification catalyst.

As described in more detail below, the carbonaceous feedstock can comprise any of a number of carbonaceous materials. For example, in one embodiment of the invention, the carbonaceous feedstock comprise one or more of anthracite, bituminous coal, sub-bituminous coal, lignite, petroleum coke, asphaltenes, liquid petroleum residues or biomass.

As described in more detail below, in certain embodiments of the invention, the carbonaceous feedstock is loaded with a gasification catalyst (i.e., to form a catalyzed carbonaceous feedstock) prior to its introduction into the catalytic gasifier. For example, the whole of the carbonaceous feedstock can be loaded with catalysts, or only part of the carbonaceous feedstock can be loaded with catalyst. Of course, in other embodiments of the invention, the carbonaceous feedstock is not loaded with a gasification catalyst before it is introduced into the catalytic gasifier.

As described in more detail below, in certain embodiments of the invention the carbonaceous feedstock is loaded with an amount of an alkali metal gasification catalyst sufficient to provide a ratio of alkali metal atoms to carbon atoms ranging from about 0.01 to about 0.10.

In certain embodiments of the invention, the carbonaceous feedstock, gasification catalyst and first gas stream are introduced into a plurality of catalytic gasifiers. For example, a single thermal reformer can supply the first gas stream to a plurality of gasifiers. In certain embodiments of the invention, a single thermal reformer can provide sufficient carbon monoxide, hydrogen and superheated steam to run catalytic gasifications in more than one catalytic gasifier. The second gas streams emerging from the separate catalytic gasifiers can be then further treated separately, or can be recombined at any point in the downstream process.

As the person of skill in the art will appreciate, the processes described herein can be performed, for example, as continuous processes or batch processes.

In certain embodiments of the invention, as shown in FIGS. 1 and 2, the process is a once-through process. In a “once-through” process, there exists no recycle of carbon-based gas into the gasifier from any of the gas streams downstream from the catalytic gasifier. However, in other embodiments of the invention, the process can include a recycle carbon-based gas stream. For example, a methane-containing stream (taken from, e.g., a second gas stream, a third gas stream or a methane product stream) can be reformed in the thermal reformer to form the first gas stream which can be admitted to the catalytic gasifier along with the carbonaceous feedstock and the gasification catalyst. In continuous operation, however, it is desirable to operate the process as a “once-through” process.

The processes of the present invention can be practiced without the use of a carbon fuel-fired superheater. Accordingly, in certain embodiments of the invention, no carbon fuel-fired superheater is present.

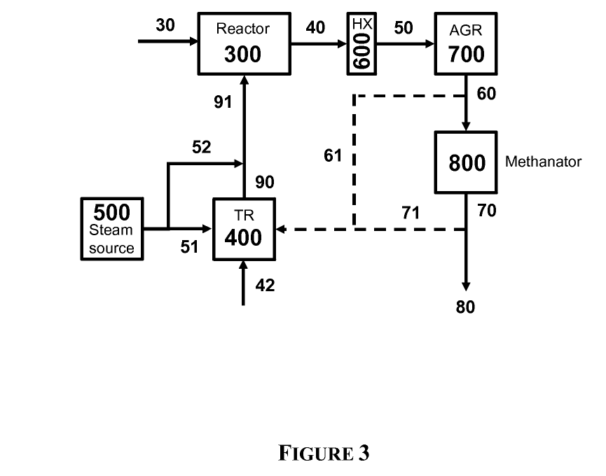

In the preceding described processes, the methane provided to the thermal reformer (400) can comprise a portion of any methane-containing gas stream which is generated by the acid gas removal process or any subsequent process. In one specific embodiment, as shown in FIG. 3, the methane provided to the thermal reformer (400), when methanation is performed subsequent to acid gas removal, can comprise a portion (71) of the methane-enriched third gas stream (70) and/or methane product stream (80); a portion (61) of the third gas stream (60); and mixtures thereof. In certain other examples, the methane provided to the thermal reformer (400) is a portion (71) of the methane-enriched third gas stream (70). In another particular example, the methane provided to the thermal reformer (400) is a portion (61) of the third gas stream (60).

In another specific embodiment, as shown in FIG. 4, the methane provided to the thermal reformer (400), when methanation is performed prior to acid gas removal, can comprise a portion (71) of the third gas stream (70); a portion (81) of the methane product stream (80); and mixtures thereof. In certain other examples, the methane provided to the thermal reformer (400) is a portion (71) of the third gas stream (70). In another particular example, the methane provided to the thermal reformer (400) is a portion (81) of the methane product stream (80).

The portion of any of the preceding streams provided to the thermal reformer (400) can comprise, for example, about 1-50 mol % of the stream (e.g., 1-50 mol % of one or more of the third, methane-enriched third, or methane product streams). In certain embodiments, when a portion of the methane-enriched third or methane product stream is provided to the thermal reformer, then the portion can comprise about 1-10 mol % or 2-5 mol % of the methane-enriched third or methane product stream. In certain other embodiments, when a portion of the third gas stream is provided to the thermal reformer, then the portion can comprise about 20-50 mol % or about 25-40 mol % of the third gas stream.

The invention provides systems that, in certain embodiments, are capable of generating “pipeline-quality natural gas” from the catalytic gasification of a carbonaceous feedstock. A “pipeline-quality natural gas” typically refers to a natural gas that is (1) within ±5% of the heating value of pure methane (whose heating value is 1010 btu/ft³ under standard atmospheric conditions), (2) substantially free of water (typically a dew point of about −40° C. or less), and (3) substantially free of toxic or corrosive contaminants. In some embodiments of the invention, the methane product stream described in the above processes satisfies such requirements.

Pipeline-quality natural gas can contain gases other than methane, as long as the resulting gas mixture has a heating value that is within ±5% of 1010 btu/ft³ and is neither toxic nor corrosive. Therefore, a methane product stream can comprise gases whose heating value is less than that of methane and still qualify as a pipeline-quality natural gas, as long as the presence of other gases does not lower the gas stream's heating value below 950 btu/scf (dry basis). A methane product stream can, for example, comprise up to about 4 mol % hydrogen and still serve as a pipeline-quality natural gas. Carbon monoxide has a higher heating value than hydrogen; thus, pipeline-quality natural gas could contain even higher percentages of CO without degrading the heating value of the gas stream. A methane product stream that is suitable for use as pipeline-quality natural gas preferably has less than about 1000 ppm CO.

### Preparation of Catalyzed Carbonaceous Feedstock

(a) Carbonaceous Materials Processing

Carbonaceous materials, such as biomass and non-biomass (supra), can be prepared via crushing and/or grinding, either separately or together, according to any methods known in the art, such as impact crushing and wet or dry grinding to yield one or more carbonaceous particulates. Depending on the method utilized for crushing and/or grinding of the carbonaceous material sources, the resulting carbonaceous particulates may be sized (i.e., separated according to size) to provide a processed feedstock as the carbonaceous feedstock or for use in a catalyst loading processes to form a catalyzed carbonaceous feedstock.

Any method known to those skilled in the art can be used to size the particulates. For example, sizing can be performed by screening or passing the particulates through a screen or number of screens. Screening equipment can include grizzlies, bar screens, and wire mesh screens. Screens can be static or incorporate mechanisms to shake or vibrate the screen. Alternatively, classification can be used to separate the carbonaceous particulates. Classification equipment can include ore sorters, gas cyclones, hydrocyclones, rake classifiers, rotating trommels or fluidized classifiers. The carbonaceous materials can be also sized or classified prior to grinding and/or crushing.

The carbonaceous particulate can be supplied as a fine particulate having an average particle size of from about 25 microns, or from about 45 microns, up to about 2500 microns, or up to about 500 microns. One skilled in the art can readily determine the appropriate particle size for the carbonaceous particulates. For example, when a fluid bed catalytic gasifier is used, such carbonaceous particulates can have an average particle size which enables incipient fluidization of the carbonaceous materials at the gas velocity used in the fluid bed catalytic gasifier.

Additionally, certain carbonaceous materials, for example, corn stover and switchgrass, and industrial wastes, such as saw dust, either may not be amenable to crushing or grinding operations, or may not be suitable for use in the catalytic gasifier, for example due to ultra fine particle sizes. Such materials may be formed into pellets or briquettes of a suitable size for crushing or for direct use in, for example, a fluid bed catalytic gasifier. Generally, pellets can be prepared by compaction of one or more carbonaceous material, see for example, previously incorporated US2009/0218424A1. In other examples, a biomass material and a coal can be formed into briquettes as described in U.S. Pat. No. 4,249,471, U.S. Pat. No. 4,152,119 and U.S. Pat. No. 4,225,457. Such pellets or briquettes can be used interchangeably with the preceding carbonaceous particulates in the following discussions.

Additional feedstock processing steps may be necessary depending on the qualities of carbonaceous material sources. Biomass may contain high moisture contents, such as green plants and grasses, and may require drying prior to crushing. Municipal wastes and sewages also may contain high moisture contents which may be reduced, for example, by use of a press or roll mill (e.g., U.S. Pat. No. 4,436,028). Likewise, non-biomass such as high-moisture coal, can require drying prior to crushing. Some caking coals can require partial oxidation to simplify catalytic gasifier operation. Non-biomass feedstocks deficient in ion-exchange sites, such as anthracites or petroleum cokes, can be pre-treated to create additional ion-exchange sites to facilitate catalyst loading and/or association. Such pre-treatments can be accomplished by any method known to the art that creates ion-exchange capable sites and/or enhances the porosity of the feedstock (see, for example, previously incorporated U.S. Pat. No. 4,468,231 and GB 1599932). Oxidative pre-treatment can be accomplished using any oxidant known to the art.

The ratio of the carbonaceous materials in the carbonaceous particulates can be selected based on technical considerations, processing economics, availability, and proximity of the non-biomass and biomass sources. The availability and proximity of the sources for the carbonaceous materials can affect the price of the feeds, and thus the overall production costs of the catalytic gasification process. For example, the biomass and the non-biomass materials can be blended in at about 5:95, about 10:90, about 15:85, about 20:80, about 25:75, about 30:70, about 35:65, about 40:60, about 45:55, about 50:50, about 55:45, about 60:40, about 65:35, about 70:20, about 75:25, about 80:20, about 85:15, about 90:10, or about 95:5 by weight on a wet or dry basis, depending on the processing conditions.

Significantly, the carbonaceous material sources, as well as the ratio of the individual components of the carbonaceous particulates, for example, a biomass particulate and a non-biomass particulate, can be used to control other material characteristics of the carbonaceous particulates. Non-biomass materials, such as coals, and certain biomass materials, such as rice hulls, typically include significant quantities of inorganic matter including calcium, alumina and silica which form inorganic oxides (i.e., ash) in the catalytic gasifier. At temperatures above about 500° C. to about 600° C., potassium and other alkali metals can react with the alumina and silica in ash to form insoluble alkali aluminosilicates. In this form, the alkali metal is substantially water-insoluble and inactive as a catalyst. To prevent buildup of the residue in the catalytic gasifier, a solid purge of char comprising ash, unreacted carbonaceous material, and various alkali metal compounds (both water soluble and water insoluble) can be routinely withdrawn.

In preparing the carbonaceous particulates, the ash content of the various carbonaceous materials can be selected to be, for example, about 20 wt % or less, or about 15 wt % or less, or about 10 wt % or less, or about 5 wt % or less, depending on, for example, the ratio of the various carbonaceous materials and/or the starting ash in the various carbonaceous materials. In other embodiments, the resulting the carbonaceous particulates can comprise an ash content ranging from about 5 wt %, or from about 10 wt %, to about 20 wt %, or to about 15 wt %, based on the weight of the carbonaceous particulate. In other embodiments, the ash content of the carbonaceous particulate can comprise less than about 20 wt %, or less than about 15 wt %, or less than about 10 wt %, or less than about 8 wt %, or less than about 6 wt % alumina, based on the weight of the ash. In certain embodiments, the carbonaceous particulates can comprise an ash content of less than about 20 wt %, based on the weight of processed feedstock where the ash content of the carbonaceous particulate comprises less than about 20 wt % alumina, or less than about 15 wt % alumina, based on the weight of the ash.

Such lower alumina values in the carbonaceous particulates allow for, ultimately, decreased losses of alkali catalysts in the catalytic gasification portion of the process. As indicated above, alumina can react with alkali source to yield an insoluble char comprising, for example, an alkali aluminate or aluminosilicate. Such insoluble char can lead to decreased catalyst recovery (i.e., increased catalyst loss), and thus, require additional costs of make-up catalyst in the overall gasification process.

Additionally, the resulting carbonaceous particulates can have a significantly higher % carbon, and thus btu/lb value and methane product per unit weight of the carbonaceous particulate. In certain embodiments, the resulting carbonaceous particulates can have a carbon content ranging from about 75 wt %, or from about 80 wt %, or from about 85 wt %, or from about 90 wt %, up to about 95 wt %, based on the combined weight of the non-biomass and biomass.

In one example, a non-biomass and/or biomass is wet ground and sized (e.g., to a particle size distribution of from about 25 to about 2500 μm) and then drained of its free water (i.e., dewatered) to a wet cake consistency. Examples of suitable methods for the wet grinding, sizing, and dewatering are known to those skilled in the art; for example, see previously incorporated US2009/0048476A1. The filter cakes of the non-biomass and/or biomass particulates formed by the wet grinding in accordance with one embodiment of the present disclosure can have a moisture content ranging from about 40% to about 60%, or from about 40% to about 55%, or below 50%. It will be appreciated by one of ordinary skill in the art that the moisture content of dewatered wet ground carbonaceous materials depends on the particular type of carbonaceous materials, the particle size distribution, and the particular dewatering equipment used. Such filter cakes can be thermally treated, as described herein, to produce one or more reduced moisture carbonaceous particulates which are passed to the catalyst loading unit operation.

Each of the one or more carbonaceous particulates can have a unique composition, as described above. For example, two carbonaceous particulates can be utilized, where a first carbonaceous particulate comprises one or more biomass materials and the second carbonaceous particulate comprises one or more non-biomass materials. Alternatively, a single carbonaceous particulate comprising one or more carbonaceous materials utilized.

(b) Catalyst Loading

The one or more carbonaceous particulates are further processed to associate at least one gasification catalyst, typically comprising a source of at least one alkali metal, to generate the catalyzed carbonaceous feedstock (30).

The carbonaceous particulate provided for catalyst loading can be either treated to form a catalyzed carbonaceous feedstock (30) which is passed to the catalytic gasifier (300), or split into one or more processing streams, where at least one of the processing streams is associated with a gasification catalyst to form at least one catalyst-treated feedstock stream. The remaining processing streams can be, for example, treated to associate a second component therewith. Additionally, the catalyst-treated feedstock stream can be treated a second time to associate a second component therewith. The second component can be, for example, a second gasification catalyst, a co-catalyst, or other additive.

In one example, the primary gasification catalyst (e.g., a potassium and/or sodium source) can be provided to the single carbonaceous particulate, followed by a separate treatment to provide one or more co-catalysts and additives (e.g., a calcium source) to the same single carbonaceous particulate to yield the catalyzed carbonaceous feedstock (30). For example, see previously incorporated US2009/0217590A1 and US2009/0217586A1. The gasification catalyst and second component can also be provided as a mixture in a single treatment to the single carbonaceous particulate to yield the catalyzed carbonaceous feedstock (30).

When one or more carbonaceous particulates are provided for catalyst loading, then at least one of the carbonaceous particulates is associated with a gasification catalyst to form at least one catalyst-treated feedstock stream. Further, any of the carbonaceous particulates can be split into one or more processing streams as detailed above for association of a second or further component therewith. The resulting streams can be blended in any combination to provide the catalyzed carbonaceous feedstock (30), provided at least one catalyst-treated feedstock stream is utilized to form the catalyzed feedstock stream.

In one embodiment, at least one carbonaceous particulate is associated with a gasification catalyst and optionally, a second component. In another embodiment, each carbonaceous particulate is associated with a gasification catalyst and optionally, a second component.

Any methods known to those skilled in the art can be used to associate one or more gasification catalysts with any of the carbonaceous particulates and/or processing streams. Such methods include but are not limited to, admixing with a solid catalyst source and impregnating the catalyst onto the processed carbonaceous material. Several impregnation methods known to those skilled in the art can be employed to incorporate the gasification catalysts. These methods include but are not limited to, incipient wetness impregnation, evaporative impregnation, vacuum impregnation, dip impregnation, ion exchanging, and combinations of these methods.

In one embodiment, an alkali metal gasification catalyst can be impregnated into one or more of the carbonaceous particulates and/or processing streams by slurrying with a solution (e.g., aqueous) of the catalyst in a loading tank. When slurried with a solution of the catalyst and/or co-catalyst, the resulting slurry can be dewatered to provide a catalyst-treated feedstock stream, again typically, as a wet cake. The catalyst solution can be prepared from any catalyst source in the present processes, including fresh or make-up catalyst and recycled catalyst or catalyst solution. Methods for dewatering the slurry to provide a wet cake of the catalyst-treated feedstock stream include filtration (gravity or vacuum), centrifugation, and a fluid press.

One particular method suitable for combining a coal particulate and/or a processing stream comprising coal with a gasification catalyst to provide a catalyst-treated feedstock stream is via ion exchange as described in previously incorporated US2009/0048476A1. Catalyst loading by ion exchange mechanism can be maximized based on adsorption isotherms specifically developed for the coal, as discussed in the incorporated reference. Such loading provides a catalyst-treated feedstock stream as a wet cake. Additional catalyst retained on the ion-exchanged particulate wet cake, including inside the pores, can be controlled so that the total catalyst target value can be obtained in a controlled manner. The catalyst loaded and dewatered wet cake may contain, for example, about 50 wt % moisture. The total amount of catalyst loaded can be controlled by controlling the concentration of catalyst components in the solution, as well as the contact time, temperature and method, as can be readily determined by those of ordinary skill in the relevant art based on the characteristics of the starting coal.

In another example, one of the carbonaceous particulates and/or processing streams can be treated with the gasification catalyst and a second processing stream can be treated with a second component (see previously incorporated US2007/0000177A1).

The carbonaceous particulates, processing streams, and/or catalyst-treated feedstock streams resulting from the preceding can be blended in any combination to provide the catalyzed carbonaceous feedstock, provided at least one catalyst-treated feedstock stream is utilized to form the catalyzed carbonaceous feedstock (30). Ultimately, the catalyzed carbonaceous feedstock (30) is passed onto the catalytic gasifier(s) (300).

Generally, each catalyst loading unit comprises at least one loading tank to contact one or more of the carbonaceous particulates and/or processing streams with a solution comprising at least one gasification catalyst, to form one or more catalyst-treated feedstock streams. Alternatively, the catalytic component may be blended as a solid particulate into one or more carbonaceous particulates and/or processing streams to form one or more catalyst-treated feedstock streams.

Typically, the gasification catalyst is present in the catalyzed carbonaceous feedstock in an amount sufficient to provide a ratio of alkali metal atoms to carbon atoms in the particulate composition ranging from about 0.01, or from about 0.02, or from about 0.03, or from about 0.04, to about 0.10, or to about 0.08, or to about 0.07, or to about 0.06.

With some feedstocks, the alkali metal component may also be provided within the catalyzed carbonaceous feedstock to achieve an alkali metal content of from about 3 to about 10 times more than the combined ash content of the carbonaceous material in the catalyzed carbonaceous feedstock, on a mass basis.

Suitable alkali metals are lithium, sodium, potassium, rubidium, cesium, and mixtures thereof. Particularly useful are potassium sources. Suitable alkali metal compounds include alkali metal carbonates, bicarbonates, formates, oxalates, amides, hydroxides, acetates, or similar compounds. For example, the catalyst can comprise one or more of sodium carbonate, potassium carbonate, rubidium carbonate, lithium carbonate, cesium carbonate, sodium hydroxide, potassium hydroxide, rubidium hydroxide or cesium hydroxide, and particularly, potassium carbonate and/or potassium hydroxide.

Optional co-catalysts or other catalyst additives may be utilized, such as those disclosed in the previously incorporated references.

The one or more catalyst-treated feedstock streams that are combined to form the catalyzed carbonaceous feedstock typically comprise greater than about 50%, greater than about 70%, or greater than about 85%, or greater than about 90% of the total amount of the loaded catalyst associated with the catalyzed carbonaceous feedstock (30). The percentage of total loaded catalyst that is associated with the various catalyst-treated feedstock streams can be determined according to methods known to those skilled in the art.

Separate carbonaceous particulates, catalyst-treated feedstock streams, and processing streams can be blended appropriately to control, for example, the total catalyst loading or other qualities of the catalyzed carbonaceous feedstock (30), as discussed previously. The appropriate ratios of the various stream that are combined will depend on the qualities of the carbonaceous materials comprising each as well as the desired properties of the catalyzed carbonaceous feedstock (30). For example, a biomass particulate stream and a catalyzed non-biomass particulate stream can be combined in such a ratio to yield a catalyzed carbonaceous feedstock (30) having a predetermined ash content, as discussed previously.

Any of the preceding catalyst-treated feedstock streams, processing streams, and processed feedstock streams, as one or more dry particulates and/or one or more wet cakes, can be combined by any methods known to those skilled in the art including, but not limited to, kneading, and vertical or horizontal mixers, for example, single or twin screw, ribbon, or drum mixers. The resulting catalyzed carbonaceous feedstock (30) can be stored for future use or transferred to one or more feed operations for introduction into the catalytic gasifiers. The catalyzed carbonaceous feedstock can be conveyed to storage or feed operations according to any methods known to those skilled in the art, for example, a screw conveyer or pneumatic transport.

Further, excess moisture can be removed from the catalyzed carbonaceous feedstock (30). For example, the catalyzed carbonaceous feedstock (30) may be dried with a fluid bed slurry drier (i.e., treatment with superheated steam to vaporize the liquid), or the solution thermally evaporated or removed under a vacuum, or under a flow of an inert gas, to provide a catalyzed carbonaceous feedstock having a residual moisture content, for example, of about 10 wt % or less, or of about 8 wt % or less, or about 6 wt % or less, or about 5 wt % or less, or about 4 wt % or less.

Optional Supplemental Gasification Processes

(a) Catalyst Recovery

Reaction of the catalyzed carbonaceous feedstock (30) under the described conditions generally provides the second gas stream (40) and a solid char product from the catalytic gasifier. The solid char product typically comprises quantities of unreacted carbonaceous material and entrained catalyst. The solid char product can be removed from the reaction chamber for sampling, purging, and/or catalyst recovery via a char outlet.

The term “entrained catalyst” as used herein means chemical compounds comprising an alkali metal component. For example, “entrained catalyst” can include, but is not limited to, soluble alkali metal compounds (such as alkali carbonates, alkali hydroxides, and alkali oxides) and/or insoluble alkali compounds (such as alkali aluminosilicates). The nature of catalyst components associated with the char extracted from a catalytic gasifier and methods for their recovery are discussed below, and in detail in previously incorporated US2007/0277437A1, US2009/0165383A1, US2009/0165382A1, US2009/0169449A1 and US2009/0169448A1.

The solid char product can be periodically withdrawn from each of the catalytic gasifiers through a char outlet which is a lock hopper system, although other methods are known to those skilled in the art. Methods for removing solid char product are well known to those skilled in the art. One such method taught by EP-A-0102828, for example, can be employed.

Char from the catalytic gasifier may be passed to a catalytic recovery unit, as described below. Such char may also be split into multiple streams, one of which may be passed to a catalyst recovery unit, and another which may be used as a methanation catalyst (as described above) and not treated for catalyst recovery.

In certain embodiments, the alkali metal in the entrained catalyst in the solid char product withdrawn from the reaction chamber of the catalytic gasifier can be recovered, and any unrecovered catalyst can be compensated by a catalyst make-up stream. The more alumina and silica that is in the feedstock, the more costly it is to obtain a higher alkali metal recovery.

In one embodiment, the solid char product from the catalytic gasifiers can be quenched with a recycle gas and water to extract a portion of the entrained catalyst. The recovered catalyst can be directed to the catalyst loading processes for reuse of the alkali metal catalyst. The depleted char can, for example, be directed to any one or more of the feedstock preparation operations for reuse in preparation of the catalyzed feedstock, combusted to power one or more steam generators (such as disclosed in previously incorporated US2009/0165376A1 and US2009/0217585A1), or used as such in a variety of applications, for example, as an absorbent (such as disclosed in previously incorporated US2009/0217582A1).

Other particularly useful recovery and recycling processes are described in U.S. Pat. No. 4,459,138, as well as previously incorporated US2007/0277437A1, US2009/0165383A1, US2009/0165382A1, US2009/0169449A1 and US2009/0169448A1. Reference can be had to those documents for further process details.

The recycle of catalyst can be to one or a combination of catalyst loading processes. For example, all of the recycled catalyst can be supplied to one catalyst loading process, while another process utilizes only makeup catalyst. The levels of recycled versus makeup catalyst can also be controlled on an individual basis among catalyst loading processes.

(b) Gas Purification

Product purification may comprise, for example, optional trace contaminant removal, ammonia removal and recovery, and sour shift processes. The acid gas removal (supra) may be, for example, performed on the cooled second gas stream (50) passed directly from a heat exchanger, or on a cooled second gas stream that has passed through either one or more of (i) one or more of the trace contaminants removal units; (ii) one or more sour shift units; (iii) one or more ammonia recovery units and (iv) the sulfur-tolerant catalytic methanators as discussed above.

(1) Trace Contaminant Removal

As is familiar to those skilled in the art, the contamination levels of the gas stream, e.g, cooled second gas stream (50), will depend on the nature of the carbonaceous material used for preparing the catalyzed carbonaceous feed stock. For example, certain coals, such as Illinois #6, can have high sulfur contents, leading to higher COS contamination; and other coals, such as Powder River Basin coals, can contain significant levels of mercury which can be volatilized in the catalytic gasifier.

COS can be removed from a gas stream, e.g., the cooled second gas stream (50), by COS hydrolysis (see, U.S. Pat. No. 3,966,875, U.S. Pat. No. 4,011,066, U.S. Pat. No. 4,100,256, U.S. Pat. No. 4,482,529 and U.S. Pat. No. 4,524,050), passing the cooled second gas stream through particulate limestone (see, U.S. Pat. No. 4,173,465), an acidic buffered CuSO₄ solution (see, U.S. Pat. No. 4,298,584), an alkanolamine absorbent such as methyldiethanolamine, triethanolamine, dipropanolamine, or diisopropanolamine, containing tetramethylene sulfone (sulfolane, see, U.S. Pat. No. 3,989,811); or counter-current washing of the cooled second gas stream with refrigerated liquid CO₂ (see, U.S. Pat. No. 4,270,937 and U.S. Pat. No. 4,609,388).

HCN can be removed from a gas stream (e.g., the cooled second gas stream (50)) by reaction with ammonium sulfide or polysulfide to generate CO₂, H₂S and NH₃ (see, U.S. Pat. No. 4,497,784, U.S. Pat. No. 4,505,881 and U.S. Pat. No. 4,508,693), or a two stage wash with formaldehyde followed by ammonium or sodium polysulfide (see, U.S. Pat. No. 4,572,826), absorbed by water (see, U.S. Pat. No. 4,189,307), and/or decomposed by passing through alumina supported hydrolysis catalysts such as MoO₃, TiO₂ and/or ZrO₂ (see, U.S. Pat. No. 4,810,475, U.S. Pat. No. 5,660,807 and U.S. Pat. No. 5,968,465).

Elemental mercury can be removed from a gas stream (e.g., the cooled second gas stream (50)) by absorption by carbon activated with sulfuric acid (see, U.S. Pat. No. 3,876,393), absorption by carbon impregnated with sulfur (see, U.S. Pat. No. 4,491,609), absorption by a H₂S-containing amine solvent (see, U.S. Pat. No. 4,044,098), absorption by silver or gold impregnated zeolites (see, U.S. Pat. No. 4,892,567), oxidation to HgO with hydrogen peroxide and methanol (see, U.S. Pat. No. 5,670,122), oxidation with bromine or iodine containing compounds in the presence of SO₂ (see, U.S. Pat. No. 6,878,358), oxidation with a H, Cl and O— containing plasma (see, U.S. Pat. No. 6,969,494), and/or oxidation by a chlorine-containing oxidizing gas (e.g., ClO, see, U.S. Pat. No. 7,118,720).

When aqueous solutions are utilized for removal of any or all of COS, HCN and/or Hg, the waste water generated in the trace contaminants removal units can be directed to a waste water treatment unit.

When present, a trace contaminant removal of a particular trace contaminant should remove at least a substantial portion (or substantially all) of that trace contaminant from the so-treated gas stream (e.g., cooled second gas stream (50)), typically to levels at or lower than the specification limits of the desired product stream. Typically, a trace contaminant removal should remove at least 90%, or at least 95%, or at least 98%, of COS, HCN and/or mercury from a cooled second gas stream.

(2) Sour Shift

A gas stream (e.g, the cooled second gas stream (50)) also can be subjected to a water-gas shift reaction in the presence of an aqueous medium (such as steam) to convert a portion of the CO to CO₂ and to increase the fraction of H₂. In certain examples, the generation of increased hydrogen content can be utilized to form a hydrogen product gas which can be separated from methane as discussed below. In certain other examples, a sour shift process may be used to adjust the carbon monoxide:hydrogen ratio in a gas stream (e.g., the cooled second gas stream (50)) for providing to a subsequent methanator. The water-gas shift treatment, for instance, may be performed on the cooled second gas stream passed directly from the heat exchanger or on the cooled second gas stream that has passed through a trace contaminants removal unit.

A sour shift process is described in detail, for example, in U.S. Pat. No. 7,074,373. The process involves adding water, or using water contained in the gas, and reacting the resulting water-gas mixture adiabatically over a steam reforming catalyst. Typical steam reforming catalysts include one or more Group VIII metals on a heat-resistant support.

Methods and reactors for performing the sour gas shift reaction on a CO-containing gas stream are well known to those of skill in the art. Suitable reaction conditions and suitable reactors can vary depending on the amount of CO that must be depleted from the gas stream. In some embodiments, the sour gas shift can be performed in a single stage within a temperature range from about 100° C., or from about 150° C., or from about 200° C., to about 250° C., or to about 300° C., or to about 350° C. In these embodiments, the shift reaction can be catalyzed by any suitable catalyst known to those of skill in the art. Such catalysts include, but are not limited to, Fe₂O₃-based catalysts, such as Fe₂O₃—Cr₂O₃ catalysts, and other transition metal-based and transition metal oxide-based catalysts. In other embodiments, the sour gas shift can be performed in multiple stages. In one particular embodiment, the sour gas shift is performed in two stages. This two-stage process uses a high-temperature sequence followed by a low-temperature sequence. The gas temperature for the high-temperature shift reaction ranges from about 350° C. to about 1050° C. Typical high-temperature catalysts include, but are not limited to, iron oxide optionally combined with lesser amounts of chromium oxide. The gas temperature for the low-temperature shift ranges from about 150° C. to about 300° C., or from about 200° C. to about 250° C. Low-temperature shift catalysts include, but are not limited to, copper oxides that may be supported on zinc oxide or alumina. Suitable methods for the sour shift process are described in previously incorporated U.S. patent application Ser. No. 12/415,050.

Steam shifting is often carried out with heat exchangers and steam generators to permit the efficient use of heat energy. Shift reactors employing these features are well known to those of skill in the art. An example of a suitable shift reactor is illustrated in previously incorporated U.S. Pat. No. 7,074,373, although other designs known to those of skill in the art are also effective. Following the sour gas shift procedure, the one or more cooled second gas streams each generally contains CH₄, CO₂, H₂, H₂S, NH₃, and steam.

In some embodiments, it will be desirable to remove a substantial portion of the CO from a cooled gas stream, and thus convert a substantial portion of the CO. “Substantial” conversion in this context means conversion of a high enough percentage of the component such that a desired end product can be generated. Typically, streams exiting the shift reactor, where a substantial portion of the CO has been converted, will have a carbon monoxide content of about 250 ppm or less CO, and more typically about 100 ppm or less CO.

In other embodiments, it will be desirable to convert only a portion of the CO so as to increase the fraction of H₂ for a subsequent methanation (e.g., a trim methanation), which will typically require an H₂/CO molar ratio of about 3 or greater, or greater than about 3, or about 3.2 or greater.

(3) Ammonia Recovery

As is familiar to those skilled in the art, gasification of biomass and/or utilizing air as an oxygen source for the catalytic gasifier can produce significant quantities of ammonia in the product gas stream. Optionally, a gas stream (e.g., the cooled second gas stream (50)) can be scrubbed by water in one or more ammonia recovery units to recovery ammonia. The ammonia recovery treatment may be performed, for example, on the cooled second gas stream passed directly from the heat exchanger or on a gas stream (e.g., the cooled second gas stream (50)) that has passed through either one or both of (i) one or more of the trace contaminants removal units; and (ii) one or more sour shift units.

After scrubbing, the gas stream (e.g., the cooled second gas stream (50)) can comprise at least H₂S, CO₂, CO, H₂ and CH₄. When the cooled gas stream has previously passed through a sour shift unit, then, after scrubbing, the gas stream can comprise at least H₂S, CO₂, H₂ and CH₄.

Ammonia can be recovered from the scrubber water according to methods known to those skilled in the art, can typically be recovered as an aqueous solution (e.g., 20 wt %). The waste scrubber water can be forwarded to a waste water treatment unit.

When present, an ammonia removal process should remove at least a substantial portion (and substantially all) of the ammonia from the scrubbed stream (e.g., the cooled second gas stream (50)). “Substantial” removal in the context of ammonia removal means removal of a high enough percentage of the component such that a desired end product can be generated. Typically, an ammonia removal process will remove at least about 95%, or at least about 97%, of the ammonia content of a scrubbed second gas stream.

(c) Methane Removal

The third gas stream or methane-enriched third gas stream can be processed, when necessary, to separate and recover CH₄ by any suitable gas separation method known to those skilled in the art including, but not limited to, cryogenic distillation and the use of molecular sieves or gas separation (e.g., ceramic) membranes. For example, when a sour shift process is present, the third gas stream may contain methane and hydrogen which can be separated according to methods familiar to those skilled in the art, such as cryogenic distillation.

Other gas purification methods include via the generation of methane hydrate as disclosed in previously incorporated U.S. patent application Ser. Nos. 12/395,330, 12/415,042 and 12/415,050.

(d) Power Generation

A portion of the steam generated by the steam source may be provided to one or more power generators, such as a steam turbine, to produce electricity which may be either utilized within the plant or can be sold onto the power grid. High temperature and high pressure steam produced within the gasification process may also be provided to a steam turbine for the generation of electricity. For example, the heat energy captured at a heat exchanger in contact with the second gas stream (40) can be utilized for the generation of steam which is provided to the steam turbine.

(e) Waste Water Treatment

Residual contaminants in waste water resulting from any one or more of the trace removal, sour shift, ammonia removal, and/or catalyst recovery processes can be removed in a waste water treatment unit to allow recycling of the recovered water within the plant and/or disposal of the water from the plant process according to any methods known to those skilled in the art. Such residual contaminants can comprise, for example, phenols, CO, CO₂, H₂S, COS, HCN, ammonia, and mercury. For example, H₂S and HCN can be removed by acidification of the waste water to a pH of about 3, treating the acidic waste water with an inert gas in a stripping column, increasing the pH to about 10 and treating the waste water a second time with an inert gas to remove ammonia (see U.S. Pat. No. 5,236,557). H₂S can be removed by treating the waste water with an oxidant in the presence of residual coke particles to convert the H₂S to insoluble sulfates which may be removed by flotation or filtration (see U.S. Pat. No. 4,478,425). Phenols can be removed by contacting the waste water with a carbonaceous char containing mono- and divalent basic inorganic compounds (e.g., the solid char product or the depleted char after catalyst recovery, supra) and adjusting the pH (see U.S. Pat. No. 4,113,615). Phenols can also be removed by extraction with an organic solvent followed by treatment of the waste water in a stripping column (see U.S. Pat. No. 3,972,693, U.S. Pat. No. 4,025,423 and U.S. Pat. No. 4,162,902).

(f) Multi-Train Processes

In the processes of the invention, each process may be performed in one or more processing units. For example, one or more catalytic gasifiers may be supplied with the carbonaceous feedstock from one or more catalyst loading and/or feedstock preparation unit operations. Similarly, the second gas streams generated by one or more catalytic gasifiers may be processed or purified separately or via their combination at a heat exchanger, sulfur-tolerant catalytic methanator, acid gas removal unit, trim methanator, and/or methane removal unit depending on the particular system configuration, as discussed, for example, in previously incorporated U.S. patent application Ser. Nos. 12/492,467, 12/492,477, 12/492,484, 12/492,489 and 12/492,497.

In certain embodiments, the processes utilize two or more catalytic gasifiers (e.g., 2-4 catalytic gasifiers). In such embodiments, the processes may contain divergent processing units (i.e., less than the total number of catalytic gasifiers) prior to the catalytic gasifiers for ultimately providing the catalyzed carbonaceous feedstock to the plurality of catalytic gasifiers and/or convergent processing units (i.e., less than the total number of catalytic gasifiers) following the catalytic gasifiers for processing the plurality of second gas streams generated by the plurality of catalytic gasifiers.

For example, the processes may utilize (i) divergent catalyst loading units to provide the catalyzed carbonaceous feedstock to the catalytic gasifiers; (ii) divergent carbonaceous materials processing units to provide a carbonaceous particulate to the catalyst loading units; (iii) convergent heat exchangers to accept a plurality of second gas streams from the catalytic gasifiers; (iv) convergent sulfur-tolerant methanators to accept a plurality of cooled second gas streams from the heat exchangers; (v) convergent acid gas removal units to accept a plurality of cooled second gas streams from the heat exchangers or methane-enriched second gas streams from the sulfur-tolerant methanators, when present; or (vi) convergent catalytic methanators or trim methanators to accept a plurality of third gas streams from acid gas removal units. As described above, in certain embodiments of the invention, a single thermal reformer can divergently supply the first gas stream to a plurality of catalytic gasification reactors.

When the systems contain convergent processing units, each of the convergent processing units can be selected to have a capacity to accept greater than a 1/n portion of the total gas stream feeding the convergent processing units, where n is the number of convergent processing units. For example, in a process utilizing 4 catalytic gasifiers and 2 heat exchangers for accepting the 4 second gas streams from the catalytic gasifiers, the heat exchanges can be selected to have a capacity to accept greater than ½ of the total gas volume (e.g., ½ to ¾) of the 4 second gas streams and be in communication with two or more of the catalytic gasifiers to allow for routine maintenance of the one or more of the heat exchangers without the need to shut down the entire processing system.

Similarly, when the systems contain divergent processing units, each of the divergent processing units can be selected to have a capacity to accept greater than a 1/m portion of the total feed stream supplying the convergent processing units, where m is the number of divergent processing units. For example, in a process utilizing 2 catalyst loading units and a single carbonaceous material processing unit for providing the carbonaceous particulate to the catalyst loading units, the catalyst loading units, each in communication with the carbonaceous material processing unit, can be selected to have a capacity to accept ½ to all of the total volume of carbonaceous particulate from the single carbonaceous material processing unit to allow for routine maintenance of one of the catalyst loading units without the need to shut down the entire processing system.

### EXAMPLES

### Example 1

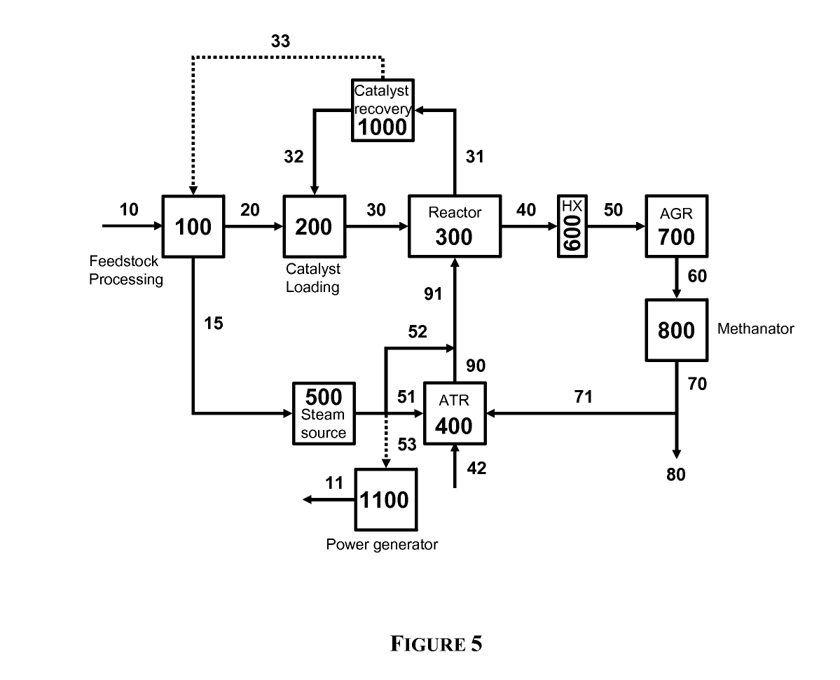

One embodiment of the processes of the invention is illustrated in FIG. 5. Therein, a carbonaceous feedstock (10) is provided to a feedstock processing unit (100) and is converted to a carbonaceous particulate (20) having an average particle size of less than about 2500 μm. The carbonaceous particulate (20) is provided to a catalyst loading unit (200) wherein the particulate is contacted with a solution comprising a gasification catalyst in a loading tank, the excess water removed by filtration, and the resulting wet cake dried with a drier to provide a catalyzed carbonaceous feedstock (30). The catalyzed carbonaceous feedstock is provided a catalytic gasifier (300).

In the catalytic gasifier, the catalyzed carbonaceous feedstock (30) is contacted with a first gas stream (91) comprising carbon monoxide, hydrogen, and superheated steam under conditions suitable to convert the feedstock a second gas stream (40) comprising at least methane, carbon dioxide, carbon monoxide, hydrogen, and hydrogen sulfide. The catalytic gasifier generates a solid char product (31), comprising entrained catalyst, which is periodically removed from their respective reaction chambers and directed to the catalyst recovery operation (1000) where the entrained catalyst (32) is recovered and returned to the catalyst loading operation (200). Depleted char (33) generated by the recovery process can be directed to the feedstock processing unit (100).

The first gas stream (91) is provided by mixing a portion (52) of the steam generated by a steam source (500) with a hot gas stream (90) generated from an autothermal reformer (400) supplied with methane (71), an oxygen-rich gas (42), and a portion of the steam (51) from the steam source (500). Fines (15) generated in the grinding or crushing process of the feedstock processing unit (100) can be provided to the steam source for combustion. Separately, a second portion (53) of the steam generated by the steam source (500) is directed to a steam turbine (1100) to generate electricity (11).

The second gas stream (40) is provided to a heat exchanger unit (600) to generate a cooled second gas stream (50). The cooled second gas stream (50) is provided to an acid gas removal unit (700) in which hydrogen sulfide and carbon dioxide in the stream are removed by sequential absorption by contacting the stream with H₂S and CO₂ absorbers, and to ultimately generate a third gas stream (60) comprising carbon monoxide, hydrogen, and methane.

The third gas stream (60) is provided to a catalytic methanator in which carbon monoxide and hydrogen present in the third gas stream are converted to methane to generate a methane-enriched third gas stream (70). A portion (71) of the methane-enriched third gas stream continuously supplies the methane for the autothermal reformer (400); the remaining portion is the methane product stream (80).

### Example 2

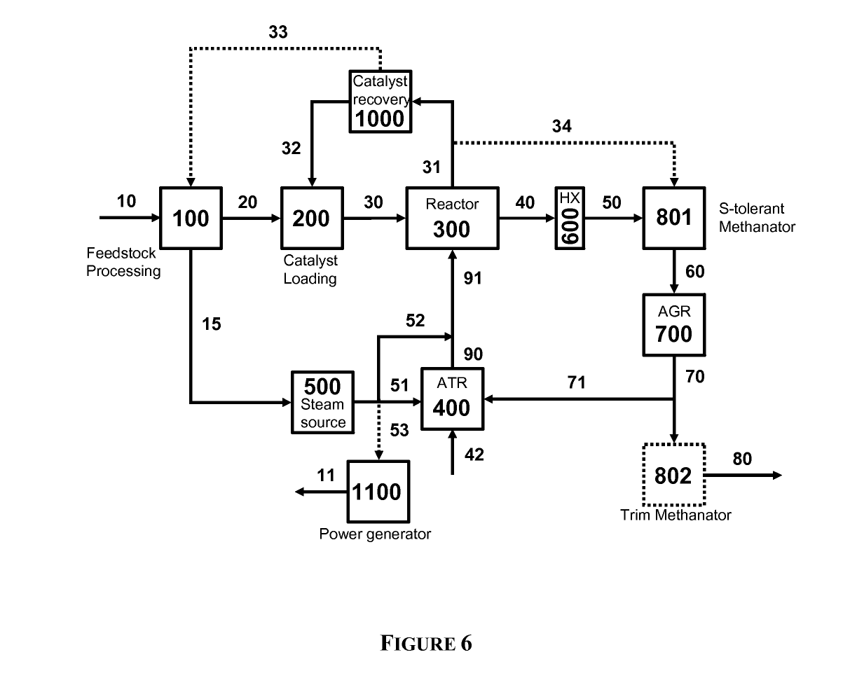

Another embodiment of the processes of the invention is illustrated in FIG. 6. Therein, a carbonaceous feedstock (10) is provided to a feedstock processing unit (100) and is converted to a carbonaceous particulate (20) having an average particle size of less than about 2500 μm. The carbonaceous particulate (20) is provided to a catalyst loading unit (200) wherein the particulate is contacted with a solution comprising a gasification catalyst in a loading tank, the excess water removed by filtration, and the resulting wet cake dried with a drier to provide a catalyzed carbonaceous feedstock (30). The catalyzed carbonaceous feedstock is provided a catalytic gasifier (300).

In the catalytic gasifier (300), the catalyzed carbonaceous feedstock (30) is contacted with a first gas stream (91) comprising carbon monoxide, hydrogen, and superheated steam under conditions suitable to convert the feedstock a second gas stream (40) comprising at least methane, carbon dioxide, carbon monoxide, hydrogen, and hydrogen sulfide. The catalytic gasifier generates a solid char product (31), comprising entrained catalyst, which is periodically removed from their respective reaction chambers and directed to the catalyst recovery operation (1000) in which entrained catalyst (32) is recovered and returned to the catalyst loading operation (200). Depleted char (33) generated by the recovery process can be directed to the feedstock processing unit (100).

The first gas stream (91) is provided by mixing a portion (52) of the steam generated by a steam source (500) with a hot gas stream (90) generated from an autothermal reformer (400) supplied with methane (71), an oxygen-rich gas (42), and a portion of the steam (51) from the steam source (500). Fines (15) generated in the grinding or crushing process of the feedstock processing unit (100) can be provided to the steam source for combustion. Separately, a second portion (53) of the steam generated by the steam source (500) is directed to a steam turbine (1100) to generate electricity.

The second gas stream (40) is provided to a heat exchanger unit (600) to generate a cooled second gas stream (50). The cooled second gas stream (50) is provided to a sulfur-tolerant methanator (801) in which carbon monoxide and hydrogen present in the cooled second gas stream (50) are reacted in the presence of a sulfur-tolerant methanation catalyst to generate a methane-enriched second gas stream (60) comprising methane, hydrogen sulfide, carbon dioxide, residual carbon monoxide and residual hydrogen. The sulfur-tolerant methanation catalyst is provided to the sulfur-tolerant methanator from a portion (34) of the char generated from the catalytic gasifier (300).

The methane-enriched second gas stream (60) is provided to an acid gas removal unit (700) in which hydrogen sulfide and carbon dioxide present in the stream are removed by sequential absorption by contacting the stream with H₂S and CO₂ absorbers, and to ultimately generate a third gas stream (70) comprising methane, residual carbon monoxide, and residual hydrogen. The third gas stream (70) is provided to a catalytic trim methanator (802) where the residual carbon monoxide and residual hydrogen in the third gas stream are converted to methane to generate a methane-enriched third gas stream (80). A portion (71) of the third gas stream continuously supplies the methane for the autothermal reformer (400); the remaining portion is provided to the trim methanator (802) to generate the methane product stream (80).

We claim:

1. A process for generating a plurality of gaseous products from a carbonaceous feedstock, and recovering a methane product stream, the process comprising the steps of:
(a) supplying methane, an oxygen-rich gas stream and steam to a thermal reformer, the reformer in communication with a catalytic gasifier;
(b) reforming a substantial portion of the methane supplied to the thermal reformer, in the presence of the oxygen-rich gas and under suitable temperature and pressure, to generate a first gas stream comprising hydrogen, carbon monoxide and superheated steam;
(c) introducing a carbonaceous feedstock, a gasification catalyst and the first gas stream to a catalytic gasifier;
(d) reacting the carbonaceous feedstock and the first gas stream in the catalytic gasifier in the presence of the gasification catalyst under suitable temperature and pressure to form a second gas stream comprising a plurality of gaseous products comprising methane, carbon dioxide, hydrogen, carbon monoxide and hydrogen sulfide;
(e) optionally reacting at least a portion of the carbon monoxide and at least a portion of the hydrogen present in the second gas stream in a catalytic methanator in the presence of a sulfur-tolerant methanation catalyst to produce a methane-enriched second gas stream;
(f) removing a substantial portion of the carbon dioxide and a substantial portion of the hydrogen sulfide from the second gas stream (or the methane-enriched second gas stream if present) to produce a third gas stream comprising a substantial portion of the methane from the second gas stream (or the methane-enriched second gas stream if present);
(g) optionally, if the third gas stream comprises hydrogen and greater than about 100 ppm carbon monoxide, reacting the carbon monoxide and hydrogen present in the third gas stream in a catalytic methanator in the presence of a methanation catalyst to produce a methane-enriched third gas stream; and
(h) recovering the third gas stream (or the methane-enriched third gas stream if present),
wherein (i) at least one of step (e) and step (g) is present, and (ii) the third gas stream (or the methane-enriched third gas stream if present) is the methane product stream, or the third gas stream (or the methane-enriched third gas stream if present) is purified to generate the methane product stream.

2. The process of claim 1, wherein steps (a), (b), (c), (d), (f) and (h), and when present (e) and (g), are continuous.

3. The process of claim 1, wherein the carbonaceous feedstock comprises one or more of anthracite, bituminous coal, sub-bituminous coal, lignite, petroleum coke, asphaltenes, liquid petroleum residues or biomass.

4. The process of claim 1, wherein the gasification catalyst comprises an alkali metal gasification catalyst.

5. The process of claim 1, wherein the carbonaceous feedstock is loaded with a gasification catalyst prior to introduction into the gasifier.

6. The process of claim 4, wherein the carbonaceous feedstock is loaded with an amount of an alkali metal gasification catalyst sufficient to provide a ratio of alkali metal atoms to carbon atoms ranging from about 0.01 to about 0.10.

7. The process of claim 1, wherein the carbonaceous feedstock, gasification catalyst and first gas stream are introduced into a plurality of catalytic gasifiers.

8. The process of claim 7, wherein a single thermal reformer supplies the first gas stream to the plurality of catalytic gasifiers.

9. The process claim 1, further comprising the step of recycling a portion of the third gas stream, or the methane-enriched third gas stream if present, or the methane product stream if it is different from the third gas or the methane-enriched third gas stream, to the thermal reformer.

10. The process of claim 9, wherein the methane supplied to the thermal reformer is the portion of the methane product stream recycled to the thermal reformer.

11. The process of claim 1, which is a once-through process.

12. The process of claim 1, wherein no carbon fuel-fired superheater is present.

13. The process of claim 1, wherein the thermal reformer is an autothermal reformer.

14. The process of claim 1, wherein the thermal reformer is a partial oxidation reactor.

15. The process of claim 1, wherein the hydrogen and carbon monoxide are present in the first gas stream in a molar ratio of about 3:1.

16. The process of claim 1, wherein the second gas stream comprises at least about 20 mol % methane based on the moles of methane, carbon dioxide, carbon monoxide and hydrogen in the second gas stream.

17. The process of claim 1, wherein the second gas stream comprises at least about 50 mol % methane plus carbon dioxide, based on the moles of methane, carbon dioxide, carbon monoxide and hydrogen in the second gas stream.

18. The process of claim 1, wherein the methane product stream is a pipeline-quality natural gas.

19. The process of claim 1, wherein step (g) is present.

20. The process of claim 1, wherein a solid char product is produced in step (d), which is periodically withdrawn from the catalytic gasifier and passed to a catalyst recovery unit.
